(19) United States
(12) Patent Application Publication
(10) Pub. No.: US 2013/0281566 A1
(43) Pub. Date: Oct. 24, 2013

(54) Paste-Like Bone Cement

(71) Applicant: Heraeus Medical GmbH, Wehrheim (DE)

(72) Inventor: Sebastian Vogt, Erfurt (DE)

(21) Appl. No.: 13/753,203

(22) Filed: Jan. 29, 2013

(30) Foreign Application Priority Data
Jan. 30, 2012 (DE) 102012001637.9

(51) Int. Cl. A61K 47/32

(52) U.S. Cl. CPC A61K 47/32
USPC 523/116; 252/182.14

(57) ABSTRACT

The present invention relates to a kit comprising a paste A and a paste B, whereby

(a) paste A contains
(a1) at least one monomer for radical polymerisation; and
(a2) at least one barbituric acid derivative as polymerisation initiator;
   (b) paste B contains
(b1) at least one monomer for radical polymerisation;
(b2) at least one heavy metal compound as polymerisation accelerator that is selected from the group consisting of heavy metal salts and heavy metal complexes;
(b4) at least one alkali or alkaline earth halide; and
(b5) at least one complexing agent for the alkali ions or alkaline earth ions (b4) that contains at least two ether groups; and whereby at least one of the pastes A and B contains, as component (a3) and/or (b3), at least one filling agent that is insoluble in (a1) and/or (b1), respectively.

The present invention relates to a kit, the use of the kit for producing a paste for mechanical fixation of articular endoprostheses, for covering skull defects, for filling bone cavities, for femuroplasty, for vertebroplasty, for kyphoplasty, for the manufacture of spacers or for the production of carrier materials for local antibiotics therapy, an initiator system, a polymerisable composition, and a form body.

Conventional polymethylmethacrylate bone cements (PMMA bone cements) have been known for decades and are based on the ground-breaking work of Sir Charnley (Charnley, J.: “Anchor-age of the femoral head prosthesis of the shaft of the femur”; J. Bone Joint Surg. 42 (1960) 28-30). The basic structure of PMMA bone cements has remained the same ever since. PMMA bone cements consist of a liquid monomer component and a powder component. The monomer component generally contains (i) the monomer, methylmethacrylate, and (ii) an activator (e.g. N,N-dimethyl-p-toluidine) dissolved therein. The powder component comprises (i) one or more polymers that are made by polymerisation, preferably by suspension polymerisation, based on methylmethacrylate and co-monomers, such as styrene, methylacrylate or similar monomers, (ii) a radio-opaquer, and (iii) an initiator, (e.g. dibenzoylperoxide). Mixing the powder component and the monomer component, the polymers of the powder component in the methylmethacrylate swell which generates a dough that can be shaped plastically. Simultaneously, the activator, N,N-dimethyl-p-toluidine, reacts with dibenzoylperoxide which disintegrates and forms radicals in the process. The radicals thus formed trigger the radical polymerisation of the methylmethacrylate. Upon advancing polymerisation of the methylmethacrylate, the viscosity of the cement dough increases until the cement dough solidifies and thus is cured.

The essential disadvantage of the previous PMMA bone cements for the medical user is that the user needs to mix the liquid monomer component and the powder component in a mixing system or in crucibles right before applying the cement. Mixing errors can easily occur in the process and adversely affect the quality of the cement. Moreover, the components must be mixed rapidly. In this context, it is important to mix all of the cement powder and monomer component without forming lumps and prevent the introduction of air bubbles during the mixing process. Unlike manual mixing, the use of vacuum mixing systems prevents the formation of air bubbles in the cement dough to a large extent. Examples of mixing systems are disclosed in patent specifications U.S. Pat. No. 4,015,945, EP-A-0 674 888, and JP 2003-181270. However, vacuum mixing systems necessitate an additional vacuum pump and are therefore relatively expensive. Moreover, depending on the type of cement concerned, a certain waiting time is required after mixing the monomer component and the powder component until the cement dough is tack-free and can be applied. Because of the large variety of errors that can occur while mixing conventional PMMA bone cements, appropriately trained personnel is required for this purpose. The corresponding training is associated with considerable expenses. Moreover, mixing of the liquid monomer component and the powder component is associated with exposure of the user to monomer vapours and particles released from the powder-like cement.

Paste-like polymethylmethacrylate bone cements have been described as an alternative to the conventional powder-liquid polymethylmethacrylate bone cements in unexamined German patent applications DE-A-10 2007 052 116, DE-A-10 2007 050 762, and DE-A-10 2007 050 763. Said bone cements are provided to the user in the form of pre-mixed pastes that are stable during storage. Said pastes each contain one methacrylate monomer for radical polymerisation, one polymer that is soluble in said methacrylate polymer, and one particulate polymer that is insoluble in said methacrylate monomer (since both pastes contain an insoluble particulate polymer, systems of this type are called “symmetrical”). In addition, one of said pastes contains a radical polymerisation initiator, whereas the other paste comprises a polymerisation activator. As a result of the selected composition, the bone cement produced from said pastes possesses sufficiently high viscosity and cohesion in order to withstand the pressure from bleeding until it is fully cured. When the two pastes are mixed, the polymerisation initiator reacts with the accelerator to form radicals that initiate the radical polymerisation of the methacrylate monomers.

Used with conventional PMMA bone cements that consisted of a powder component and a monomer liquid, the initiator system of dibenzoylperoxide and N,N-dimethyl-p-toluidine has proven its value in general (K.-D. Kuhn: Knochenzemente für die Endoprothetik: ein aktueller Vergleich der physikalischen and chemischen Eigenschaften handelsiiblicher PMMA-Zemente. Springer-Verlag Berlin Heidelberg N.Y., 2001). In this context, dibenzolyperoxide is present as a solid in the cement powder and N,N-dimethyl-p-toluidine is dissolved in the monomer compo-nent.

However, our experiments with cement pastes using the dibenzoylperoxide/N,N-dimethyl-p-toluidine initiator system demonstrated that pastes containing N,N-dimethyl-p-toluidine have a pronounced tendency to polymerise spontaneously. Moreover, the accelerator, N,N-dimethyl-p-toluidine, which has proven its value with conventional powder/liquid polymethylmethacrylate bone cements, has been the subject of some criticism due to its toxicological properties.

Aside from these redox systems, initiator systems based on the use of barbiturates have also been described. Accordingly, Del. -A-10 2007 050 762 and DE -A-10 2007 050 763 describe an initia-tor system comprising alkaline earth salts of barbituric acid derivatives, halide ion donors and copper compounds. In this context, alkaline earth salts of barbiturates and basic copper salts are contained in a paste. These two salts are insoluble in the methacrylate monomer. A weak organic acid such as 2-ethylhexanoic acid is present in a second paste. In addition, a chloride ion donor is present in the pastes as well, whereby tetraalkylammoniumchloride is preferably used as chloride ion donor according to the teaching of DE -A-10 2007 050 763. Mixing the two pastes, the weak organic acid simultaneously converts both the barbiturate into the soluble acid form and copper into a soluble copper salt. The advantage of this system, in particular in the case of pastes with multi-functional monomers, is that earlier diffusion and ion exchange processes allow the proc-essing time to be increased which otherwise is very short, usually on the order of seconds, where multi-functional monomers are used. However, it is disadvantageous that quarternary ammonium chlorides can occasionally trigger spontaneous polymerisation in the presence of dissolved heavy metal ions. It has therefore proven to be advantageous to develop an initiator system based on barbiturates, heavy metal ions, and chloride ions, whereby the chloride ions in the form of inor-ganic salts rather than the tetraalkylammonium chlorides known from the prior art are used. An initiator system of this type is described, for example, in DE 10 2010 024 653 A1. Sodium chlo-ride, potassium chloride, and calcium chloride do not dissolve in the common hydrophobic monomer that is used for bone cements, such as methylmethacrylate. Lithium chloride dissolves to some extent in methylmethacrylate. However, in the presence of traces of water, basically non-reproducible changes of the initiation behaviour may occur.

The present invention was based on the object to overcome the disadvantages of the prior art concerning bone cement systems that are based on at least two pastes.

The present invention was based, in particular, on the object to provide a kit based on two pastes, whereby the pastes, while they are separated from each other, should feature the highest possible stability against polymerisation (i.e. should show as little tendency to undergo spontaneous po-lymerisation as possible).

The present invention was also based on the object to provide a kit based on two pastes and/or an initiator system, in which alkali chlorides and alkaline earth chlorides can be dissolved safely in hydrophobic methacrylate monomers even in the presence of traces of water. The kit should be characterised by an initiation behaviour that is as reproducible as possible.

A kit comprising a paste A and a paste B contributes to a solution meeting the object specified above,

whereby
(a) paste A contains
(a1) at least one monomer for radical polymerisation; and
(a2) at least one barbituric acid derivative as polymerisation initiator;
   (b) paste B contains
(b1) at least one monomer for radical polymerisation;
(b2) at least one heavy metal compound as polymerisation accelerator that is selected from the group consisting of heavy metal salts and heavy metal complexes;
(b4) at least one alkali or alkaline earth halide; and
(b5) at least one complexing agent for the alkali ions or alkaline earth ions (b4) that contains at least two ether groups;
   and whereby at least one of the pastes A and B contains, as component (a3) and/or (b3), at least one filling agent that is insoluble in (a1) and/or (b1), respectively.

The invention is further based on the idea to use a first paste that contains a barbiturate that is soluble in a monomer for radical polymerisation, such as methylmethacrylate. Mixing said first paste with a second paste containing a heavy metal compound, an alkali or alkaline earth halide, and a complexing agent for the alkali ions or alkaline earth ions aside from the monomer for radical polymerisation, the soluble barbiturate reacts with the preferably basic heavy metal salt due to its acidity. It has been found surprisingly that this makes it feasible to initiate the polymerisation reaction in the presence of an inorganic halide ion donor that is preferably soluble in the monomer. The action of the barbiturate on the preferably basic heavy metal salt obviously converts the heavy metal ions into a soluble salt form which initiates polymerisation of the methacrylate monomer through its action on the barbituric acid. Using the complexing component (b5) in paste B ensures that sufficient amounts of the alkali/alkaline earth halide (n4) dissolve in the monomer (b1) for radical polymerisation of paste B at all times, which ensures that the initiation behaviour is reproducible.

According to the invention, a kit shall be understood to be a system made up of at least two components. Although reference to two components (i.e. paste A and paste B) is made in the following, the kit can just as well contain more than two components, for example three, four, five or more than five components, according to need. The individual components preferably are provided to be packaged separate from each other such that the ingredients of the one kit component do not contact the ingredients of another kit component. Accordingly, it is feasible, for example, to package the respective kit components separate from each other and to store them together in a reservoir container.

Paste A contains, as component (a1), a monomer for radical polymerisation, whereby this is preferably a monomer that is liquid at a temperature of 25° C. and a pressure of 1,013 hPa.

Preferably, the monomer (a1) for radical polymerisation is a methacrylate monomer, in particular a methacrylic acid ester. Preferably, the methacrylic acid ester (a1) is a monofunctional methacrylic acid ester. Preferably, said substance is hydrophobic. The use of hydrophobic monofunctional methacrylic acid esters (a1) allows later enlargement of the volume of the bone cement due to the uptake of water and thus damage to the bone to be prevented. According to a preferred embodiment, the monofunctional methacrylic acid ester is hydrophobic if it contains no further polar groups aside from the ester group. The monofunctional hydrophobic methacrylic acid ester preferably comprises no carboxyl groups, hydroxyl groups, amide groups, sulfonic acid groups, sulfate groups, phosphate groups or phosphonate groups.

The esters preferably are alkyl esters. According to the invention, cycloalkyl esters are also included in alkyl esters. According to a preferred embodiment, the alkyl esters are esters of methacrylic acid and alcohols comprising 1 to 20 carbon atoms, more preferably 1 to 10 carbon atoms, even more preferably 1 to 6 carbon atoms, and particularly preferably 1 to 4 carbon atoms. The alcohols can be substituted or non-substituted and preferably are non-substituted. Moreover, the alcohols can be saturated or unsaturated and preferably are saturated.

According to a particularly preferred embodiment, the monomer (a1) for radical polymerisation is a methacrylic acid methylester, methacrylic acid ethylester or a mixture of said two monomers.

According to a further preferred embodiment, the monomer (a1) for radical polymerisation is not a bisphenol A-derived methacrylic acid ester.

The monomer (a1) for radical polymerisation used according to the invention preferably has a molar mass of less than 1,000 g/mol. This also comprises monomers for radical polymerisation that are components of a mixture of monomers, whereby at least one of the monomers for radical polymerisation of the mixture of monomers has a defined structure with a molar mass of less than 1,000 g/mol.

The monomer (a1) for radical polymerisation is preferably characterised in that an aqueous solution of the monomer (a1) for radical polymerisation has a pH in the range of 5 to 9, preferably in the range of 5.5 to 8.5, even more preferably in the range of 6 to 8, and particularly preferably in the range of 6.5 to 7.5.

Paste A preferably contains 15 to 85% by weight, more preferably 20 to 70% by weight, even more preferably 25 to 60% by weight, and particularly preferably 25 to 50% by weight, each relative to the total weight of paste A, of the at least one monomer (a1) for radical polymerisation.

Moreover, paste A contains, as component (a2), at least one barbituric acid derivative as polymerisation initiator, whereby said barbituric acid derivative is preferably a barbituric acid derivative that is selected from the group consisting of 1-mono-substituted barbiturates, 5-mono-substituted barbiturates, 1,5-di-substituted barbiturates, 1,3,5-tri-substituted barbiturates, and 1,3,5-tetra-substituted barbiturates. Of these, those that cannot cross the blood-brain barrier at all or in pharmacologically insignificant amounts at most are preferred. Accordingly, 1-mono-substituted barbiturates, 5-mono-substituted barbiturates, 1,5-di-substituted barbiturates, and 1,3,5-tri-substituted barbiturates are particularly preferred barbituric acid derivatives in this context with 1,5-disubstituted barbiturates and 1,3,5-tetra-substituted barbiturates being most preferred.

According to a preferred embodiment, the barbituric acid derivative (a2) is soluble in the polymerisable monomer (a1). The barbituric acid derivative (a2) is soluble in the polymerisable monomer (a1) if at least 1 g/l, preferably at least 3 g/l, even more preferably at least 5 g/l, and particularly preferably at least 10 g/l of the barbituric acid derivative (a3) dissolve(s) in the polymerisable monomer (a1) at a temperature of 25° C.

There is no limitation with regard to the type of substituents on the barbituric acid. The substituents can, for example, be aliphatic or aromatic substituents. In this context, alkyl, cycloalkyl, allyl or aryl substituents can be preferred. The substituents can also include hetero atoms. In particular, the substituents can be thiol substituents. Accordingly, 1,5-disubstituted thiobarbiturates or 1,3,5-trisubstituted thiobarbiturates can be preferred.

According to a preferred embodiment, the substituents each have a length of 1 to 10 carbon atoms, more preferably a length of 1 to 8 carbon atoms, and particularly preferably a length in the range of 2 to 7 carbon atoms.

Barbiturates having one substituent each at position 1 and position 5, one substituent each at positions 1, 3, and 5 or one substituent each at positions 1 and 3 and two substituents at position 5 are preferred according to the invention.

According to a particularly preferred embodiment, the barbituric acid derivative (a2) is selected from the group consisting of 1-cyclohexyl-5-ethyl-barbituric acid, 1-phenyl-5-ethyl-barbituric acid, and 1,3,5-trimethyl-barbituric acid.

Preferably, paste A contains an amount of the at least one barbituric acid derivative (a2) in a range of 0.1 to 10% by weight, more preferably in a range of 0.5 to 8% by weight, and even more preferably in a range of 1 to 5% by weight, each relative to the total weight of paste A.

Paste B also contains, as component (b1), a monomer for radical polymerisation, whereby this is preferably a monomer that is liquid at a temperature of 25° C. and a pressure of 1,013 hPa. The monomer (b1) for radical polymerisation contained in a kit can be identical to or different from the monomer (a1) for radical polymerisation, whereby it is preferred for the monomer (a1) for radical polymerisation and the monomer (b1) for radical polymerisation to be identical.

The monomer (b1) for radical polymerisation preferably is a methacrylate monomer, in particular a methacrylic acid ester. Preferably, the methacrylic acid ester (b1) is a monofunctional methacrylic acid ester. Preferably, said substance is hydrophobic. The use of hydrophobic monofunctional methacrylic acid esters (b1) allows later enlargement of the volume of the bone cement due to the uptake of water and thus damage to the bone to be prevented. According to a preferred embodiment, the monofunctional methacrylic acid ester is hydrophobic if it contains no further polar groups aside from the ester group. The monofunctional hydrophobic methacrylic acid ester preferably comprises no carboxyl groups, hydroxyl groups, amide groups, sulfonic acid groups, sulfate groups, phosphate groups or phosphonate groups.

The esters preferably are alkyl esters. According to the invention, cycloalkyl esters are also included in alkyl esters. According to a preferred embodiment, the alkyl esters are esters of methacrylic acid and alcohols comprising 1 to 20 carbon atoms, more preferably 1 to 10 carbon atoms, even more preferably 1 to 6 carbon atoms, and particularly preferably 1 to 4 carbon atoms. The alcohols can be substituted or non-substituted and preferably are non-substituted. Moreover, the alcohols can be saturated or unsaturated and preferably are saturated.

According to a particularly preferred embodiment, the monomer (b1) for radical polymerisation is a methacrylic acid methylester, methacrylic acid ethylester or a mixture of said two monomers.

According to a further particularly preferred embodiment, the monomer (b1) for radical polymerisation is not a bisphenol A-derived methacrylic acid ester.

The monomer (b1) for radical polymerisation used according to the invention preferably has a molar mass of less than 1,000 g/mol. This also comprises monomers for radical polymerisation that are components of a mixture of monomers, whereby at least one of the monomers for radical polymerisation of the mixture of monomers has a defined structure with a molar mass of less than 1,000 g/mol.

The monomer (b1) for radical polymerisation is characterised in that an aqueous solution of the monomer (b1) for radical polymerisation has a pH in the range of 5 to 9, preferably in the range of 5.5 to 8.5, even more preferably in the range of 6 to 8, and particularly preferably in the range of 6.5 to 7.5.

Paste B preferably contains 15 to 85% by weight, more preferably 20 to 70% by weight, even more preferably 25 to 60% by weight, and particularly preferably 25 to 50% by weight, each relative to the total weight of paste B, of the at least one monomer (b1) for radical polymerisation.

Paste B further contains, as component (b2), at least one heavy metal compound selected from the group consisting of heavy metal salts and heavy metal complexes, as polymerisation accelerator, where it has proven to be particularly advantageous for the at least one heavy metal compound (b2) to be poorly soluble, preferably even insoluble, in the monomer (b1) for radical polymerisation. A heavy metal compound (b2) is considered to be poorly soluble or insoluble in the monomer (b1) for radical polymerisation if less than 1 g/l, preferably less than 0.1 g/l, even more preferably less than 0.01 g/l, yet more preferably less than 0.001 g/l, even yet more preferably less than 0.0001 g/l, and most preferably no significant amounts of the heavy metal compound (b2) at all dissolve at a temperature of 25° C. in the monomer (b1) for radical polymerisation (i.e. the heavy metal compound (b2) is insoluble in the monomer (b1) for radical polymerisation).

According to the invention, heavy metal compounds shall be understood to mean metals with a density of at least 3.5, preferably of at least 5, at a temperature of 20° C.

According to a preferred embodiment, the heavy metal compound (b2) is a basic heavy metal compound. Basic heavy metal compound shall be understood to mean a heavy metal compound which, when dissolved or suspended in water, has a pH of at least 6.5, preferably at least 7, and even more preferably at least 7.5.

According to a particularly preferred embodiment, the heavy metal compounds (b2) are compounds of metals that can change their oxidation state. Copper (II), iron (II), iron (III), manganese (II), manganese (III), cobalt (II), and cobalt (III) compounds are preferred in this context according to the invention with copper(II) compounds being particularly preferred.

Provided they are heavy metal compounds that are poorly soluble or insoluble in the monomer (b1) for radical polymerisation, the heavy metal compounds according to the invention are preferably capable, in the presence of the barbituric acid derivatives (a2), of converting into a form that is soluble in the monomer (a1) and/or (b1) for radical polymerisation.

According to the invention, the heavy metal compounds (b2) are heavy metal salts or heavy meta1 complexes.

The heavy metal salts (b2) preferably are halides, hydroxides, carbonates or carboxylic acid salts of heavy metals. Copper (II), iron (II), iron (III), manganese (II), manganese (III), cobalt (II), and cobalt (III) salts are preferred heavy metals salts.

Moreover, halide salts are conceivable as heavy metal compound (b2) that is insoluble in (b1). The halide salt can preferably be selected from the group consisting of heavy metal chlorides and bromides. According to a particular embodiment, the halide salt is a compound selected from the group consisting of manganese(II) chloride, iron(II) chloride, iron(III) chloride, cobalt(II) chloride, and cobalt(III) chloride.

According to a particularly preferred embodiment, the heavy metal salt (b2) is selected from the group consisting of copper(II) hydroxide, basic copper(II) carbonate or a mixture of at least two thereof, in particular a mixture of copper(II) hydroxide and basic copper(II) carbonate.

Preferably, paste B contains an amount of the heavy metal compound (b2) in a range of 0.0005 to 0.5% by weight, more preferably in a range of 0.001 to 0.05% by weight, and particularly preferably in a range of 0.001 to 0.01% by weight, each relative to the total weight of paste B.

Moreover, paste B contains, as component (b4), at least one alkali or alkaline earth halide. In principle, F⁻, Cl⁻, and Br⁻ are conceivable as halide anion with Cl⁻ being particularly preferred. Particularly preferred alkali or alkaline earth halides include potassium chloride, sodium chlo-ride, calcium chloride, and magnesium chloride with lithium chloride being most preferred as alkali chloride (b4).

Moreover, it is also preferred in this context that paste B contains an amount of the at least one alkali or alkaline earth halide (b4) in a range of 0.001 to 7.5% by weight, more preferably in a range of 0.01 to 5% by weight, even more preferably in a range of 0.1 to 2.5% by weight, and most preferably in a range of 0.5 to 1.5% by weight, each relative to the total weight of paste B.

Paste B also contains, as component (b5), at least one complexing agent for the alkali ions or alkaline earth ions (b4) that contains at least two ether groups, whereby the at least one complexing agent (b5) is preferably selected from the group consisting of a podand, a coronand (=crown ether), a cryptand or a mixture of at least two of these.

The term, “podand”, refers to open-chain compounds bearing donor atoms, such as, for example, oxygen atoms, sulfur atoms, nitrogen atoms or phosphorus atoms, in a linear or branched chain. This means that podands do not comprise a pre-formed cavity for cations. Only upon complex formation with cations, they form cavities around the cations. The term, “coronand”, refers to cyclic polyethers containing ethane bridges that are connected to each other via oxygen atoms. Unlike podands, coronands have pre-formed cavities of defined dimensions. This allows coronands to selectively complex cations according to their ion radius. In contrast, “cryptands” are bicyclic and polycyclic polyethers that also have preformed cavities.

Complexing agents (b5) that are preferred according to the invention are selected from the group consisting of benzo-12-crown-4, cyclohexyl-12-crown-4,2,3-naphto-12-crown-4,6,6-dibenzyl-12-crown-4,6-dodecyl(14-crown-4)-6-ethanol-diethyl-phosphate, bis[(12-crown-4)-methyl]-2-dodecyl-2-methyl-malonate, and a mixture of at least two of these. Said complexing agents are well-suited specifically for selective complexing of lithium ions. The advantage of said complexing agents is that the complexing crown ether structure has a ring size of 12 atoms and in one case of 15 atoms. Therefore, alkali ions that are present in the body, such as sodium and potassium, as well as alkaline earth ions, such as calcium ions, cannot be complexed. This reduces the potential risk of toxic effects. Aside from said small coronands, hydrophobic derivatives of 18-crown-6, such as benzo-18-crown-6, cyclohexyl-18-crown-6 are suitable on principle. Other preferred complexing agents include N,N-diheptyl-N,N′,5,5-tetramethyl-3,7-dioxanonamide or 5-butyl-5-ethyl-N,N, —N′,N′-tetracyclohexyl-3,7-dioxaazelaic acid diamide.

Moreover, it is preferred according to the invention that the molar ratio of alkali or alkaline earth halide (b4) to complexing agent (b5) in paste B is at least 1:1, more preferably at least 1:1.5, even more preferably at least 1:1.8, and yet more preferably at least 1:2.0.

Moreover, paste B can contain water as further component (b6), whereby the molar ratio of water (b6) to alkali and/or alkaline earth halide (b5) preferably is at least 1:1, more preferably at least 2:1, even more preferably at least 3:1, and yet more preferably at least 4:1. The presence of water in paste B facilitates the complexing of the cations of the alkali or alkaline earth halide (b4) through the complexing agent (b5) such that the complexed cations can be transported into the monomer (b1) for radical polymerisation.

The kit according to the invention is characterised in that at least one of the pastes A and B contains, as component (a3) or (b3), at least one filling agent that is insoluble in (a1) or (b1), respectively. Provided one of the two pastes contains an insoluble filling agent and the other paste contains no insoluble filling agent at all or contains a negligible amount of insoluble filling agent as compared to the amount present in the other paste, the kit is called “asymmetrical”. In contrast, a so-called “symmetrical” kit has approximately comparable amounts of the insoluble filling agent present in both pastes.

The filling agent (a3) (in case of paste A) and/or (b3) (in case of paste B) is a solid substance at room temperature and capable of increasing the viscosity of the mixture composed of the remaining ingredients contained in paste A and/or paste B, respectively. The filling agent (a3) and/or (b3) should be biocompatible.

According to a preferred embodiment, the filling agent (a3) and/or (b3) is selected from polymers, inorganic salts, inorganic oxides, metals, and metal alloys.

Preferably, the filling agent (a3) and/or (b3) is particulate. According to a particularly preferred embodiment, the filling agent (a3) and/or (b3) has an average particle size in the range of 10 nm to 100 μm and particularly preferably in the range of 100 nm to 10 μm. The average particle size shall be understood herein to mean a size range that applies to at least 90 percent of the particles. In the scope of the invention, the term, polymers, shall include both homopolymers and copolymers.

The polymer that can be used as filling agent (a3) and/or (b3) preferably is a polymer with a mean (by weight) molar mass of at least 150,000 g/mol. The specification of the molar mass refers to the molar mass determined by viscosimetry. The polymer can, for example, be a polymer or copolymer of a methacrylic acid ester. According to a particularly preferred embodiment, the at least one polymer is selected from the group consisting of polymethacrylic acid methylester (PMMA), polymethacrylic acid ethylester (PMAE), polymethacrylic acid propylester (PMAP), polymethacrylic acid is opropylester, poly(methylmethacrylate-co-methylacrylate), and poly(styrene-co-methylmethacrylate). However, the polymer can just as well be selected from the group consisting of polyethylene, polypropylene or polybutadiene. Moreover, the polymer can be cross-linked or non-cross-linked with cross-linked polymers being preferred. In this context, the cross-linking is effected through a difunctional compound. The difunctional compound can be selected, for example, from the group consisting of alkylene glycol dimethacrylates. An expedient cross-linker is, for example, ethylene glycol dimethacrylate.

The inorganic salt that can be used as filling agent (a3) and/or (b3) can be a salt that is soluble or insoluble in the monomer (a1) and/or (b1) for radical polymerisation. Preferably, the inorganic salt is a salt of an element selected from the second main group of the periodic system of elements. According to a preferred embodiment, the inorganic salt is a calcium, strontium or barium salt. According to a particularly preferred embodiment, the inorganic salt is calcium sulfate, barium sulfate or calcium carbonate.

The inorganic oxide that can be used as filling agent (a3) and/or (b3) can preferably be a metal oxide. According to a preferred embodiment, the inorganic oxide is a transition metal oxide. According to a particularly preferred embodiment, the inorganic oxide is titanium dioxide or zirconium dioxide.

The metal that can be used as filling agent (a3) and/or (b3) can, for example, be a transition metal. According to a preferred embodiment, the metal is tantalum or tungsten.

The metal alloy that can be used as filling agent (a3) and/or (b3) is an alloy of at least two metals. Preferably, the alloy contains at least one transition metal. According to a particularly preferred embodiment, the alloy comprises at least tantalum or tungsten. The alloy can also be an alloy of tantalum and tungsten.

The filling agent (a3) and/or (b3) is insoluble in the monomer (a1) and/or (b1) for radical polymerisation, respectively. According to the invention, the filling agent (a3) and/or (b3) is insoluble in the at least one monomer (a1) and/or (b1) for radical polymerisation, if the solubility of the filling agent (a3) and/or (b3) in the monomer (a1) and/or (b1) for radical polymerisation at a temperature of 25° C. is less than 50 g/l, preferably is less than 25 g/l, more preferably is less than 10 g/l, and even more preferably is less than 5 g/l.

It is particularly preferred according to the invention that the at least one polymer that is insoluble in (a1) and/or (b1) is selected from the group consisting of cross-linked poly(methylmethacrylate-co-methylacrylate), cross-linked poly(methylmethacrylate), and a mixture of said two polymers.

Moreover, according to the invention, paste A, paste B or paste A and paste B, though particularly preferably paste A and paste B, can contain a polymer (a7) and/or (b7) that is soluble in (a1) and/or (b1), respectively. According to the invention, said polymer (a7) and/or (b7) is soluble in the polymerisable monomer contained in the paste that contains the soluble polymer as well, if at least 10 g/l, preferably at least 25 g/l, more preferably at least 50 g/l, and particularly preferably at least 100 g/l of the polymer dissolve in said polymerisable monomer. The polymer (a7) and/or (b7) that is soluble in the polymerisable monomer (a1) and/or (b1), respectively, can be a homopolymer or a copolymer. Said polymer (a7) and/or (b7) preferably is a polymer with a mean (by weight) molar mass of at least 150,000 g/mol. The polymer (a7) and/or (b7) can, for example, be a polymer or copolymer of a methacrylic acid ester. According to a particularly preferred embodiment, the at least one polymer (a7) and/or (b7) is selected from the group consisting of polymethacrylic acid methylester (PMMA), polymethacrylic acid ethylester (PMAE), polymethacrylic acid propylester (PMAP), polymethacrylic acid isopropylester, poly(methylmethacrylate-co-methylacrylate), and poly(styrene-co-methylmethacrylate).

The amount of the polymer (a7) and/or (b7) that is soluble in the monomer (a1) and/or (b1) for radical polymerisation, respectively, that is present in the paste containing said polymer depends on whether or not the corresponding paste contains a filling agent (a3) and/or (b3) that is insoluble in the monomer (a1) and/or (b1) for radical polymerisation, respectively. Usually, the amount of the polymer (a7) and/or (b7) that is soluble in the monomer (a1) and/or (b1) for radical polymerisation, respectively, that is present in the paste containing said polymer is in a range of 1 to 85% by weight, relative to the total weight of the paste containing said soluble polymer.

Pastes A and B can contain further components aside from the components explained above. Said further components can each be present either in paste A, in paste B or in paste A and paste B.

According to a preferred embodiment, at least one radio-opaquer is present in at least one of the pastes A and B. The radio-opaquer can be a common radio-opaquer in this field. Suitable radio-opaquers can be soluble or insoluble in the monomer (a1) for radical polymerisation or the monomer (b1) for radical polymerisation. The radio-opaquer is preferably selected from the group consisting of metal oxides (such as, for example, zirconium oxide), barium sulfate, toxicologically acceptable heavy metal particles (such as, for example, tantalum), ferrite, magnetite (supramagnetic magnetite also, if applicable), and biocompatible calcium salts. Said radio-opaquers preferably have a mean particle diameter in the range of 10 nm to 500 p.m. Moreover, conceivable radio-opaquers also include esters of 3,5-bis(acetamido)-2,4,6-triiodobenzoic acid, gadolinium compounds, such as gadolinium chelate involving the esters of 1,4,7,10-tetraazacyclododecan-1,4,7,10-tetraacetic acid (DOTA).

According to another preferred embodiment, at least one of the pastes A and B contains at least one colourant. The colourant can be a common colourant in this field and preferably can be a food colourant. Moreover, the colourant can be soluble or insoluble in the at least one monomer (a1) for radical polymerisation or the at least one monomer (a2) for radical polymerisation. According to a particularly preferred embodiment, the colourant is selected from the group consisting of E101, E104, E132, E141 (chlorophyllin), E142, riboflavin, and lissamine green. According to the invention, the term, colourant, shall also include colour varnishes, such as, for example, colour varnish green, the aluminium salt of a mixture of E104 and E132.

According to another preferred embodiment, at least one of the pastes A and B contains at least one pharmaceutical agent. The at least one pharmaceutical agent can be present in at least one of pastes A and B in dissolved or suspended form.

The pharmaceutical agent can preferably be selected from the group consisting of antibiotics, antiphlogistic agents, steroids, hormones, growth factors, bisphosphonates, cytostatic agents, and gene vectors. According to a particularly preferred embodiment, the at least one pharmaceutical agent is an antibiotic.

Preferably, the at least one antibiotic is selected from the group consisting of aminoglyoside antibiotics, glycopeptide antibiotics, lincosamide antibiotics, gyrase inhibitors, carbapenems, cyclic lipopeptides, glycylcyclines, oxazolidones, and polypeptide antibiotics.

According to a particularly preferred embodiment, the at least one antibiotic is a member selected from the group consisting of gentamicin, tobramycin, amikacin, vancomycin, teicoplanin, dalbavancin, lincosamine, clindamycin, moxifloxacin, levofloxacin, ofloxacin, ciprofloxacin, doripenem, meropenem, tigecycline, linezolide, eperezolide, ramoplanin, metronidazole, timidazole, omidazole, and colistin, as well as salts and esters thereof.

Accordingly, the at least one antibiotic can be selected from the group consisting of gentamicin sulfate, gentamicin hydrochloride, amikacin sulfate, amikacin hydrochloride, tobramycin sulfate, tobramycin hydrochloride, clindamycin hydrochloride, lincosamine hydrochloride, and moxifloxacin.

The at least one antiphlogistic agent is preferably selected from the group consisting of non-steroidal antiphlogistic agents and glucocorticoids. According to a particularly preferred embodiment, the at least one antiphlogistic agent is selected from the group consisting of acetylsalicylic acid, ibuprofen, diclofenac, ketoprofen, dexamethasone, prednisone, hydrocortisone, hydrocortisone acetate, and fluticasone.

The at least one hormone is preferably selected from the group consisting of serotonin, somatotropin, testosterone, and estrogen.

Preferably, the at least one growth factor is selected from the group consisting of Fibroblast Growth Factor (FGF), Transforming Growth Factor (TGF), Platelet Derived Growth Factor (PDGF), Epidermal Growth Factor (EGF), Vascular Endothelial Growth Factor (VEGF), insulin-like growth factors (IGF), Hepatocyte Growth Factor (HGF), Bone Morphogenetic Protein (BMP), interleukin-1B, interleukin 8, and nerve growth factor.

The at least one cytostatic agent is preferably selected from the group consisting of alkylating agents, platinum analogues, intercalating agents, mitosis inhibitors, taxanes, topoisomerase inhibitors, and antimetabolites.

The at least one bisphosphonate is preferably selected from the group consisting of zoledronate and aledronate.

According to another preferred embodiment, at least one of the pastes A and B contains at least one biocompatible elastomer. Preferably, the biocompatible elastomer is particulate. Preferably, the biocompatible elastomer is soluble in the at least one monomer (a1) for radical polymerisation or the at least one monomer (b1) for radical polymerisation. The use of butadiene as biocompatible elastomer has proven to be particularly well-suited.

According to another preferred embodiment, at least one of the pastes A and B contains at least one monomer with adsorption groups. An amide group can, for example, be an adsorption group. Accordingly, the monomer with adsorption group can, for example, be methacrylic acid amide. Using at least one monomer with adsorption groups would allow the binding of the bone cement to articular endoprostheses to be influenced in a targeted manner.

According to another preferred embodiment, at least one of the pastes A and B contains at least one stabiliser. The stabiliser should be suitable to prevent spontaneous polymerisation of the monomers (a1) and/or (b1) for polymerisation that are present in pastes A and B. Moreover, the stabiliser should not undergo interfering interactions with the other components contained in the pastes. Stabilisers of said type are known according to the prior art. According to a preferred embodiment, the stabiliser is 2,6-di-tert-butyl-4-methylphenol and/or 2,6-di-tert-butyl-phenol.

According to a first particular refinement of the kit according to the invention, the kit is an “asymmetrical” kit. It is preferred in this context that paste A contains 20 to 70% by weight, particularly preferably 25 to 60% by weight, even more preferably 30 to 55% by weight, and most preferably 34 to 47% by weight, each relative to the total weight of paste A, of the filling agent (a3) that is insoluble in (a1), and paste B contains less than 5% by weight, particularly preferably less than 1% by weight, even more preferably less than 0.1% by weight, and yet more preferably less than 0.01% by weight, each relative to the total weight of paste B, of the filling agent (b3) that is insoluble in (b1), whereby it is most preferred that paste B contains no filling agent (b3) that is insoluble in (b1) at all.

Moreover, in the context of said first particular refinement of the kit according to the invention, it is preferred that paste A contains an amount of a polymer (a7) that is soluble in (a1) in a range of 1 to 25% by weight, particularly preferably in a range of 2 to 20% by weight, even more preferably in a range of 2 to 18% by weight, and most preferably in a range of 3 to 16% by weight, each relative to the total weight of paste A, and paste B contains an amount of a polymer (b7) that is soluble in (b1) in a range of 25 to 85% by weight, particularly preferably in a range of 35 to 85% by weight, even more preferably in a range of 40 to 80% by weight, and most preferably in a range of 50 to 75% by weight, each relative to the total weight of paste B.

Moreover, it is preferred in the context of said first particular refinement of the kit according to the invention that the weight ratio of filling agent (b3) that is insoluble in (b1) to the at least one polymer (b7) that is soluble in (b1) is no more than 0.2, more preferably no more than 0.15, even more preferably no more than 0.1, yet more preferably no more than 0.05, particularly preferably no more than 0.02, and even more particularly preferably is equal to 0.

According to a second particular refinement of the kit according to the invention, the kit is a “symmetrical” kit. It is preferred in this context that paste A contains 15 to 85% by weight, particularly preferably 15 to 80% by weight, and even more preferably 20 to 75% by weight, each relative to the total weight of paste A, of the filling agent (a3) that is insoluble in (a1), and paste B contains 15 to 85% by weight, particularly preferably 15 to 80% by weight, and even more preferably 20 to 75% by weight, each relative to the total weight of paste B, of the filling agent (b3) that is insoluble in (b1).

Moreover, in the context of said second particular refinement of the kit according to the invention, it is preferred that paste A contains an amount of a polymer (a7) that is soluble in (a1) in a range of 5 to 50% by weight, particularly preferably in a range of 10 to 40% by weight, and even more preferably in a range of 20 to 30% by weight, each relative to the total weight of paste A, and/or paste B contains an amount of a polymer (b7) that is soluble in (b1) in a range of 5 to 50% by weight, particularly preferably in a range of 10 to 40% by weight, and even more preferably in a range of 20 to 30% by weight, each relative to the total weight of paste B.

According to the invention, the purpose of the kit containing at least pastes A and B is the production of bone cement.

For this purpose, the at least two pastes A and B are mixed with each other, upon which another paste, paste C, is obtained.

The mixing ratio preferably is 0.5 to 1.5 parts by weight of paste A and 0.5 to 1.5 parts by weight of paste B. According to a particularly preferred embodiment, the fraction of paste A is 30 to 70% by weight and the fraction of paste B is 30 to 70% by weight, each relative to the total weight of pastes A and B, respectively.

The mixing process can involve common mixing devices, for example a static mixer or a dynamic mixer.

The mixing process can proceed in a vacuum. However, the use of the initiator system according to the invention also allows for mixing of pastes A and B in the absence of a vacuum without adverse effect on the properties of the bone cement.

Paste C that is ultimately obtained after mixing the pastes of the kit is tack-free according to the ISO 5833 standard and can be processed without delay.

The bone cement generated from paste C by curing attains high strength approximately six to eight minutes after mixing the pastes contained in the kit.

According to a preferred embodiment, the kit according to the invention can be used for mechanical fixation of articular endoprostheses, for covering skull defects, for filling bone cavities, for femuroplasty, for vertebroplasty, for kyphoplasty, for the manufacture of spacers, and for the production of carrier materials for local antibiotics therapy.

In this context, the term, “spacer”, shall be understood to mean implants that can be used temporarily in the scope of the two-step exchange of prostheses in septic revision surgeries.

Carrier materials for local antibiotics therapy can be provided as spheres or sphere-like bodies or as bean-shaped bodies. Besides, it is also feasible to produce rod-shaped or disc-shaped carrier materials that contain bone cement made from the kit according to the invention. Moreover, the carrier materials can also be threaded onto absorbable or non-absorbable suture material in a bead-like manner.

The uses according to the invention of bone cement described above are known from the literature and have been described therein on numerous occasions.

According to the invention, the kit is used for the above-described uses in that, preferably, the pastes contained in the kit are mixed with each other to produce a paste that is then used in the above-described uses just like pastes known from the prior art.

Furtherore, a contribution to meeting the above-mentioned object is made by an initiator system containing

(i1) at least one barbituric acid derivative;
(i2) at least one heavy metal salt;
(i3) at least one alkali or alkaline earth halide;
(i4) at least one complexing agent for alkali or alkaline earth ions that contains at least two ether groups; and
(i5) water, if applicable.

The compounds mentioned above with regard to the kit according to the invention as preferred barbituric acid derivatives (a2), as heavy metal salt (b2), alkali or alkaline earth halides (b4), and complexing agents for alkali or alkaline earth halides (b5) are preferred as barbituric acid derivative (i1), as heavy metal salt (i2), as alkali or alkaline earth halide (i3), and as complexing agents for alkali or alkaline earth halides (i4).

A contribution to meeting the objects specified above is also made by a polymerisable composition containing at least one monomer for radical polymerisation, preferably a methacrylate monomer, and an initiator system according to the invention. A polymerisable composition of said type can be obtained, for example, through mixing pastes A and B of the kit according to the invention.

A contribution to meeting the objects specified above is also made by a form body obtained through polymerisation of the polymerisable composition according to the invention or through polymerisation of a paste that is can be obtained through mixing paste A and paste B of the kit according to the invention. Form bodies according to the scope of the present invention can be any three-dimensional body, in particular the “spacers” described above.

The invention shall be illustrated through the examples described in the following, though without limiting the scope of the invention.

### EXAMPLES

### Examples 1 to 4

### According to the Scope of the Invention

Pastes A of examples 1-4 were produced by simple mixing of the components. The pastes thus formed were then stored over night at room temperature.

| | Composition of paste A | | | |
|---|---|---|---|---|
| Educt | Example 1 | | Examples 2-4 | |
| 1-Cyclohexyl-5-ethyl-barbituric acid (a2) | 2.0 | g | 2.0 | g |
| MMA (a1) | 19.0 | g | 19.0 | g |
| Methacrylamide | 0.4 | g | 0.4 | g |
| Ethyleneglycol dimethacrylate | 0.1 | g | 0.1 | g |
| Soluble PMMA (a7) | 6.0 | g | 6.0 | g |
| Cross-linked PMMA (a3) | 15.0 | g | 12.7 | g |
| 2,6-di-tert.-butyl-4-methyl-phenol | 20 | mg | 20 | mg |

| — | — | — | |
|---|---|---|---|
| Gentamicin sulfate | — | 2.3 | g |
| (activity coefficient 628) | | | |

Pastes B were produced by first weighing the lithium chloride, water, and MMA and placing them in a vessel. The mixture was stirred at room temperature until the aqueous phase was no longer detectable by eye. Then, all other components were added and the mixture was homogenised through stirring. The resulting pastes were then stored over night at room temperature.

| | Composition of paste B | | | | | | | |
|---|---|---|---|---|---|---|---|---|
| Educt | Example 1 | | Example 2 | | Example 3 | | Example 4 | |
| Lithium chloride (b4) | 40 | mg | 40 | mg | 40 | mg | 40 | mg |
| Benzo-12-crown-4 | 317 | mg | 317 | mg | 317 | mg | 317 | mg |
| (b5) | | | | | | | | |
| Dist. water (b6) | 80 | mg | 80 | mg | 80 | mg | 80 | mg |
| 2,6-di-tert.-butyl- | 35- | mg | 35- | mg | 35- | mg | 35- | mg |
| 4-methyl-phenol | | | | | | | | |
| MMA (b1) | 21.0 | g | −21.0 | g | 21.00- | g | 21.0- | g |
| Soluble PMMA (b7) | 17.0 | g | 17.0 | g | 17.0 | g | 17.0 | g |
| Zirconium dioxide | 5.0 | g | 5.0 | g | 5.0 | g | 5.0 | g |
| Copper(II) | 6.0 | mg | 4.0 | mg | 6.0 | mg | 8.0 | mg |
| hydroxide (b2) | | | | | | | | |

The pastes A and B of each of the examples 1-4 were mixed with each other at a weight ratio of 1:1 (paste A from example 1 was mixed with paste B from example 1, etc.). This immediately resulted in pastes that were tack-free and cured after a few minutes.

The mixed pastes produced from pastes A and B of examples 1-4 were used to produce strip-shaped test bodies with dimensions of (75 mm×10 mm×3.3 mm) for the assay of bending strength and flexural modulus and cylindrical test bodies (diameter 6 mm, height 12 mm) were used for the assay of compressive strength. The test bodies were stored in water for 24 hours at 37° C. Then the 4-point bending strength, flexural modulus, and the compressive strength of the test bodies were determined using a Zwick universal testing device. Moreover, strip-shaped test bodies with dimensions of (20 mm×10 mm×3,3 mm) were produced and stored in water for 24 hours at 37° C. Then, the dynstat bending strength and the dynstat impact strength of said test bodies were assayed using a dynstat testing apparatus.

| | Examples | | | |
|---|---|---|---|---|
| | 1 | 2 | 3 | 4 |
| 4-point flexural strength | 70.0 ± 1.7 | 59.2 ± 2.2 | 59.9 ± 2.5 | 59.6 ± 1.9 |
| [MPA] | | | | |
| Flexural modulus [MPA] | 2,615 ± 59 | 2,347 ± 56 | 2,377 ± 56 | 2,204 ± 115 |
| Compressive strength | 95.3 ± 3.1 | 80.6 ± 2.5 | 82.0 ± 1.7 | 83.3 ± 3.1 |
| [MPA] | | | | |
| Dynstat flexural strength | 94.6 ± 4.3 | 70.1 ± 3.2 | 76.0 ± 3.5 | 74.1 ± 2.5 |
| [MPA] | | | | |
| Dynstat impact strength | 5.2 ± 0.3 | 3.5 ± 0.3 | 3.9 ± 0.4 | 4.0 ± 0.3 |
| [kJ/m²] | | | | |

### Examples 5-11

### According to the Scope of the Invention

Pastes A and B according to example 1 were produced, in which the complexing agent benzo-12-crown-4 was replaced by the complexing agent cyclohexyl-12-crown-4,2,3-naphto-12-crown-4 (example 5), 6,6-dibenzyl-12-crown-4,6-dodecyl(14-crown-4)-6-ethanol-diethylphosphate (example 6), bis[(12-crown-4)-methyl]-2-dodecyl-2-methyl-malonate (example 7), benzo-18-crown-6 (example 8), cyclohexyl-18-crown-6 (example 9), N,N-diheptyl-N,N′,5,5-tetramethyl-3,7-dioxanonamide (example 10) or 5-butyl-5-ethyl-N,N, —N′,N′-tetracyclohexyl-3,7-dioxaazelaic acid diamide (example 11) using, in paste B, the same molar amount that was used of benzo-12-crown-4 in example 1 of paste B.

As before, pastes A and B of examples 5-11 each were mixed with each other at a weight ratio of 1:1. This immediately resulted in pastes that were tack-free and cured after a few minutes.

### Reference Example

### Not According to the Scope of the Invention

Pastes A and B of example 1 were produced except that paste B contained no complexing agent. It was evident that mixing methylmethacrylate, water, and lithium chloride without adding complexing agent generated two phases, whereby the major amount of lithium chloride accumulated in the aqueous phase. The aqueous phase persisted even after stirring for several days. A paste B was produced regardless. But the polymerisation after mixing paste A and paste B was delayed strongly, whereby the extent of said delay was a function of the water content of paste B, amongst other factors.

1. A kit comprising a paste A and a paste B, whereby
(a) paste A comprises
(a1) at least one monomer for radical polymerisation; and
(a2) at least one barbituric acid derivative as polymerisation initiator;
(b) paste B comprises
(b1) at least one monomer for radical polymerisation;
(b2) at least one heavy metal compound as polymerisation accelerator that is selected from the group consisting of heavy metal salts and heavy metal complexes;
(b4) at least one alkali or alkaline earth halide; and
(b5) at least one complexing agent for the alkali ions or alkaline earth ions (b4) that contains at least two ether groups;
and wherein at least one of pastes A and B contains, as component (a3) or (b3), at least one filling agent that is insoluble in (a1) or (b1), respectively.

2. The kit according to claim 1, wherein the at least one monomer (a1) and/or (b1) for radical polymerisation is a methacrylate monomer.

3. The kit according to claim 1 wherein paste A and paste B contain an amount of the at least one monomer (a1) or (b1) for radical polymerisation in a range of 15 to 85% by weight, each relative to the total weight of paste A and/or paste B, respectively.

4. The kit according to claim 1 wherein the at least one barbituric acid derivative (a2) is selected from the group consisting of 1-cyclohexyl-5-ethyl-barbituric acid, 1-phenyl-5-ethyl-barbituric acid, and 1,3,5-trimethyl-barbituric acid.

5. The kit according to claim 1 wherein paste A contains an amount of the at least one barbituric acid derivative (a2) in a range of 0.1 to 10% by weight, each relative to the total weight of paste A.

6. The kit according to claim 1 wherein the at least one heavy metal compound (b2) is selected from the group consisting of copper(II) hydroxide, cobalt(II) hydroxide, and basic copper(II) carbonate.

7. The kit according to claim 1 wherein paste B contains an amount of the heavy metal compound (b2) in a range of 0.0005 to 0.5% by weight, relative to the total weight of paste B.

8. The kit according to claim 1 wherein the at least one alkali or alkaline earth halide (b4) is an alkali chloride, an alkaline earth chloride or a mixture of at least two of these.

9. The kit according to claim 1 wherein the at least one alkali halide (b4) is lithium chloride.

10. The kit according to claim 1 wherein paste B contains an amount of the alkali or alkaline earth halide (b4) in a range of 0.001 to 7.5% by weight, relative to the total weight of paste B.

11. The kit according to claim 1 wherein the at least one complexing agent (b5) is selected from the group consisting of a podand, a coronand, a cryptand or a mixture of at least two of these.

12. The kit according to claim 1 wherein the at least one complexing agent (b5) is selected from the group consisting of benzo-12-crown-4, cyclohexyl-12-crown-4,2,3-naphto-12-crown-4,6,6-dibenzyl-12-crown-4,6-dodecyl(14-crown-4)-6-ethanol-diethyl-phosphate, bis[(12-crown-4)-methyl]-2-dodecyl-2-methyl-malonate, N,N-diheptyl-N,N′,5,5-tetramethyl-3,7-dioxanonamide, 5-butyl-5-ethyl-N,N, —N′,N′-tetracyclohexyl-3,7-dioaazelaic acid diamide, and a mixture of at least two of these.

13. The kit according to claim 1 wherein the molar ratio of alkali or alkaline earth halide (b4) to complexing agent (b5) in paste B is at least 1:1.

14. The kit according to claim 1 wherein the at least one filling agent (a3) and/or (b3) that is insoluble in (a1) and/or (b1), respectively, is a particulate polymer.

15. The kit according to claim 1 wherein the at least one filling agent (a3) and/or (b3) that is insoluble in (a1) and/or (b1), respectively, is selected from the group consisting of cross-linked poly(methylmethacrylate-co-methylacrylate), cross-linked poly(methylmethacrylate), and a mixture of said two polymers.

16. The kit according to claim 1 wherein paste B contains water as further component (b6).

17. The kit according to claim 16, whereby the mass ratio of water (b6) and alkali and/or alkaline earth halide (b4) is at least 1:1.

18. The kit according to claim 1 wherein paste A, paste B or paste A and paste B contain a polymer (a7) and/or (b7) that is soluble in (a1) and/or (1)1), respectively.

19. The kit according to claim 18, whereby the polymer (a7) and/or (b7) that is soluble in (a1) and/or (1)1), respectively, is selected from the group consisting of poly(methacrylic acid methylester), poly(methacrylic acid ethylester), poly-(methylmethacrylic acid propylester), poly(methacrylic acid isopropylester), poly(methylmethacrylate-co-methylacrylate), and poly(styrene-co-methylmethacrylate).

20. A method for producing a paste for mechanical fixation of articular prostheses, for covering skull defects, for filling bone cavities, for femuroplasty, for vertebroplasty, for kyphoplasty, for the manufacture of spacers or for the production of carrier materials for local antibiotics therapy comprising using the kit according to claim 1 on a patient in need thereof.

21. An initiator system comprising
(i1) at least one barbituric acid derivative;
(i2) at least one heavy metal salt;
(i3) at least one alkali or alkaline earth halide;
(i4) at least one complexing agent for alkali or alkaline earth ions that contains at least two ether groups; and
(i5) water, if applicable.

22. A polymerisable composition containing at least one monomer for radical polymerisation and an initiator system as defined in claim 21.

23. The polymerisable composition according to claim 22 whereby the monomer for radical polymerisation is a methacrylate monomer.

24. A form body obtained through polymerisation of a polymerisable composition or through polymerisation of a paste that can be obtained through mixing paste A and paste B of the kit as defined in claim 1.
